(19) United States
(12) Patent Application Publication
(10) Pub. No.: US 2002/0176860 A1
(43) Pub. Date: Nov. 28, 2002

(54) Fusion proteins for specific treatment of cancer and autoimmune diseases

(76) Inventors: Mathieu Hubertus, M. Noteborn, Leiderdorp (NL); Johan Renes, Soest (NL); Ying-Hui Zhang, Leiden (NL)

Correspondence Address: TRASK BRITT, P.O. BOX 2550, SALT LAKE CITY, UT, 84110, US

(21) Appl. No.: 10/113,790

(22) Filed: Mar. 29, 2002

Related U.S. Application Data
(60) Provisional application No. 60/280,229, filed on Mar. 30, 2001.

(51) Int. Cl. A61K 48/00; A61K 39/395; C07K 16/46; C07K 14/00; C12N 9/16; A61K 9/127

(52) U.S. Cl. 424/178.1; 530/391.1; 435/196; 424/450; 514/8; 530/395

(57) ABSTRACT

The invention relates to the field of apoptosis. The invention provides novel therapeutic substances, for example novel therapeutic (non-)viral vectors or proteinaceous compounds, that contain apoptin in conjunction with cytotoxic agents, especially in those cases when cells are derailed, such as in cancer- and autoimmune-derived cells.

### CROSS-REFERENCE TO RELATED APPLICATION

Pursuant to the provisions of 35 U.S.C. §119(e), priority is hereby claimed from U.S. Provisional Patent Application Serial No. 60/280,229, filed Mar. 30, 2001, which is hereby incorporated herein in its entirety.

### BACKGROUND OF THE INVENTION

1. Field of the Invention

The present invention relates to the field of therapies based on molecular biology. The invention further relates to the field of treatment of cancer and/or autoimmune diseases. Where reference is made in this specification to either, the other should be included unless expressly excluded. The invention also relates to induction of apoptosis in cells associated with cancer or autoimmune diseases.

2. State of the Art

Apoptosis is an active and programmed physiological process for eliminating superfluous, altered or malignant cells (Eamshaw, 1995; Duke et al., 1996). Apoptosis is characterized by shrinkage of cells, segmentation of the nucleus, condensation and cleavage of DNA into domain-sized fragments, followed by intemucleosomal degradation in most cells. The apoptotic cells fragment into membrane-enclosed apoptotic bodies. Finally, neighboring cells and/or macrophages will rapidly phagocytose these dying cells (Wyllie et al., 1980; White, 1996). The apoptotic process can be initiated by a variety of regulatory stimuli (Wyllie, 1995; White 1996; Levine, 1997).

Changes in the cell survival rate play an important role in human pathogenesis, for example, in cancer development and autoimmune diseases, which is caused by enhanced proliferation but also by decreased cell death (Kerr et al., 1994; Paulovich, 1997). A variety of chemotherapeutic compounds and radiation have been demonstrated to induce apoptosis in tumor cells, in many instances via wild-type p53 protein (Thompson, 1995; Bellamy et al., 1995; Steller, 1995; McDonell et al., 1995).

Many tumors, however, acquire a mutation in p53 during their development, often correlating with poor response to cancer therapy. Certain transforming genes of tumorigenic DNA viruses can inactivate p53 by directly binding to it. Examples of such agents are the E6 protein from oncogenic subtypes of the Human Papiloma Virus and the large T antigen of the tumor DNA virus SV40 it (Teodoro, 1997). Another example of the emergence of a strong resistance to various apoptosis-inducing hemotherapeutic agents in (leukemic) tumors is the association of a high expression level of the proto-oncogene Bcl-2 or Bcr-abl with reduced sensitivity of these tumors to therapy (Hockenberry 1994; Sachs and Lotem, 1997).

Autoimmune diseases comprise a group of severe diseases that are characterized by inflammatory disorders, such as Crohn's disease and rheumatoid arthritis (RA). Recently, evidence has been provided that RA-related fibroblast-like synoviocytes (FLS) exhibit characteristics of transformed/tumorigenic cells. For instance, adherence to plastic or extracellular matrix is generally required for normal fibroblasts to proliferate and survive in culture for prolonged periods of time. Transformed cells, however, can grow in suspension in semi-solid medium without contact with a solid surface. While FLS typically grow and thrive under conditions that permit adherence, they can, in some circumstances, proliferate in an anchorage-independent manner (Lafyatis et al., 1989). Furthermore, the expression of several oncogenes, such as c-myc, has been reported for cultured FLS (Gay and Gay, 1989). Higher endogenous release of growth factors, such as tumor growth factor-beta and other cytokines, have also been described for FLS (Bucala et al., 1991; Remmers et al., 1990; Geiler, 1994; Firestein, 1995 and 1995a). In addition, in some cases non-functional tumor-suppressor p53 has been related with RA (Aupperle et al., 1998). Although mutant p53 is not an oncogene, it prevents induction of apoptosis by endogenous or exogenous agents. All these data indicate that FLS are irreversibly altered in RA and that an autonomous process allows them to remain activated even after removal from the articular inflammatory milieu (Firestein, 1995).

Therefore, therapies for cancer and autoimmune diseases, such as RA, that can restore the cell death process in the disease-related cells need to be developed. Among the broad array of genes that have been evaluated for tumor therapy, those encoding prodrug activation enzymes are especially appealing, as they directly complement ongoing clinical chemotherapeutic regimes. These enzymes can activate prodrugs that have a low inherent toxicity, using both bacterial and yeast enzymes, or enhance prodrug activation by mammalian enzymes. Activation of ganciclovir by viral thymidine kinase is currently being evaluated in clinical trials.

In, for example, cancer gene therapy, vectors target delivery of therapeutic genes to tumor cells by means of direct injection into the tumor mass or surrounding tissues. The activated drug is able to act on non-transduced tumor cells. This “bystander effect” can even act at distant sites and is believed to be mediated by the immune system (Aghi et al., 2000). However, several drawbacks of this approach have been reported. For example, Van der Eb et al. (1998) have reported that treatment of rats with an adenovirus synthesizing Herpes Simplex Virus thymidine kinase (HSV TK) and ganciclovir administration resulted in a severe hepatic dysfunction. These studies clearly showed that normal (non-transformed) non-mitotic tissues -can be affected by adenovirus mediated HSV TK transfer and subsequent ganciclovir treatment. Given the hepatropic nature of systemically administered adenovirus-derived vectors, it will be essential to monitor liver functions of patients included in all gene therapy trials involving adenoviral vectors with the HSV-TK gene. Furthermore, over expression of the proto-oncogene Bcl-2, which has anti-apoptotic activity, significantly increased tumor cell resistance against a large panel of cytotoxic drugs and also against HSV-TK/ganciclovir mediated gene therapy (Fels et al., 2000). Therefore, approaches that will make the HSV-TK approach based on adenoviral gene therapy less toxic are needed. Furthermore, strategies circumventing the resistance against activated prodrugs need to be developed.

Apoptin (also called “vp3”; the terms “apoptin” and “vp3” may be used interchangeably herein) is a small protein derived from chicken anemia virus (“CAV”) (Notebom and De Boer, 1996; Notebom et al., 1991, 1994 and 1998a) that induces apoptosis in human malignant and transformed cell lines. In vitro and in vivo, apoptin fails to induce programmed cell death in normal lymphoid, dermal, epidermal, endothelial and smooth muscle cells. However, when normal cells are transformed, they become susceptible to apoptosis induced by apoptin. In normal cells, apoptin was found predominantly in the cytoplasm, whereas in transformed or malignant cells, i.e. cells characterized by hyperplasia, metaplasia or dysplasia, it was located in the nucleus (Danen-van Oorschot, 1997; Notebom, 1996).

Long-term expression of apoptin in normal human fibroblasts revealed that apoptin apparently has no toxic or transforming activity in these cells (Danen-van Oorschot, 1997; Noteborn, 1996). Notebom and Pietersen (1998) and Pietersen et al. (1999) have provided evidence that adenovirus-expressed apoptin does not have an acute toxic affect in vivo, whereas in nude mice it has a strong anti-tumor activity. Further evidence of a lack of toxicity in vivo comes from transgenic mice which express apoptin from an MHC-I promoter and which have no observable abnormalities (Noteborn and Zhang, 1998). Of importance in the treatment of tumors that have become resistant to chemo or radiation therapy is that apoptin-induced apoptosis occurs in the absence of functional p53 (Zhuang et al., 1995a), and cannot be blocked by Bcl-2, Bcr-abl (Zhuang et al., 1995) or the Bcl-2-associating protein BAG-1 (Danen-van Oorschot, 1997a; Noteborn, 1996). In addition, it appears that even pre-malignant, minimally transformed cells, may be sensitive to the death-inducing affect of apoptin (Notebom and Zhang, 1998). Recently, Notebom and Pietersen (2000) provided evidence that apoptin can recognize the transformed-like autoimmune conditions (e.g., RA), which results in apoptin-induced apoptosis in RA-affected fibroblast-like synoviocytes.

### BRIEF SUMMARY OF THE INVENTION

To further enlarge the array of therapeutic anti-cancer or anti-autoimmune-disease compounds available in the art, additional therapeutic tools are desired. The invention provides novel therapeutic substances, for example novel therapeutic proteinaceous compounds that can contain apoptin jointly with other proteinaceous cytotoxic proteins or protein fragments, such as HSV TK or (non-)viral vector systems expressing these fusion proteins, especially in those cases when cells are derailed, such as cancer-derived or autoimmune-derived cells. In particular, the invention describes a therapy based on the activation of cytotoxic compounds and/or making cytotoxic compounds more specific for tumor cells and cells related to autoimmune diseases by binding it to apoptin protein.

In a first embodiment, the invention provides a fusion protein consisting of HSV TK and apoptin that induces apoptosis in a tumor-specific way. The TK-apoptin fusion protein exerts its tumor-specific cytotoxicity when administered to cells.

The invention includes a gene delivery vehicle (or vector), which enables using the features of the tumor-specific apoptin and an enzyme that can activate cytotoxic prodrugs for cancer and autoimmune disease treatment via the use of gene therapy. Such a gene delivery vehicle, which is an independently infectious vector, can, for example, be a virus, a liposome, a polymer or the like, that, in itself, can infect or in any other way deliver genetic information to, for example, tumor cells that can be treated. The genetic information comprises a nucleic acid molecule encoding apoptin-TK-like activity.

Additionally, the invention includes a gene delivery system that, in itself, is replication-defective virus but can replicate in helper or packaging cells to generate progeny gene delivery vehicles. The gene delivery vehicle thus provided by the invention can, for instance, be an adenovirus, a parvovirus, a retrovirus or other DNA or RNA recombinant viruses that can be used as delivery vehicles or a plasmovirus.

Additionally, the invention provides a gene delivery vehicle which has additionally been supplemented with a specific ligand or target molecule or molecules, by which the gene delivery vehicle can be specifically directed to deliver its genetic information at a target cell of choice. Such a target molecule or antibody is reactive with a tumor cell surface receptor or protein.

The invention furthermore includes all steps needed for the construction of a recombinant, replication-defective adenovirus expressing the TK-apoptin fusion product. High titers of recombinant-TK-apoptin adenovirus can be produced by means of adenovirus packaging cell lines, such as 293, 911 and PER.C6™. (Notebom and Pietersen, 1998). The TK-apoptin does not exhibit a detectable negative effect on all necessary adenovirus replication steps and other adenovirus life-cycle events under cell culture conditions.

Recombinant replication-defective adenovirus expresses TK-apoptin in high amounts in various tumor cells and/or cells related to autoimmune diseases, resulting in the induction of apoptosis. In contrast, expression of TK-apoptin, with or without ganciclovir treatment, in normal non-transformed human cells by means of recombinant adenovirus does not result in the induction of TK-apoptin induced cell death.

In particular, the invention relates to anti-tumor and anti-autoimmune disease therapies. Treatment of tumor cells and/or cells related to autoimmune diseases will take place by expression of TK-apoptin fusion protein by means of infecting cells with gene delivery vehicles, such as adenovirus vectors that contain a coding sequence for a protein with TK-apoptin-like activity. Therefore, the invention provides gene delivery vehicles, such as the adenovirus expressing apoptin, which is a potential anti-tumor or anti-autoimmune disease agent. Adenovirus regulation of TK-apoptin after ganciclovir treatment does not detectably induce apoptosis in human normal non-transformed cells, indicating that the toxicity of in vivo treatment with recombinant TK-apoptin adenovirus will be low.

Expression of TK-apoptin in tumor/autoimmune disease-related cells may also take place by infecting cells with other DNA and/or RNA viral vectors, besides adenovirus vectors, that contain a coding sequence for TK-apoptin. In addition, virus-derived vector systems, such as plasmoviruses, can be used for the induction of TK-apoptin induced apoptosis in tumor cells.

Moreover, TK-apoptin fusion protein or derivatives of it will also be effective against tumors that have become resistant to (chemo)-therapeutic induction of apoptosis due to the lack of functional p53 and/or (over)-expression of Bcl-2 and other apoptosis-inhibiting agents.

To eradicate a tumor, it is imperative that all cells of the tumor, including its potential mini-metastases, are removed upon treatment. Due to the bystander effect observed by infection of human tumor cells with recombinant adenovirus synthesizing TK-apoptin protein, in vivo tumor cells undergo cell death despite the fact that they were not transduced and able to produce TK-apoptin protein themselves. This will result in the complete regression of the treated tumor and, most likely, of distal tumors as well.

The invention also describes that the activity and behavior of the recombinant proteins is similar to the activity of TK-apoptin protein produced by transcription and translation of the TK-apoptin DNA. In particular, the detailed description set forth hereinafter provides evidence that microinjection of TK-apoptin fusion protein, produced in E. coli cells and purified, results in induction of apoptosis in human tumor and/or RA-derived cells but not in normal human primary cells.

The invention also has the ability to differentiate between normal and transformed cells that recombinant TK-apoptin protein harbors potential activity for the destruction of tumor cells, or other hyperplasia, metaplasia or dysplasia, with minimal or no toxicity to normal tissue. The invention also describes another example of an effective anti-tumor therapy based on apoptin-derived proteinaceous substances.

The invention describes a method allowing direct introduction of TK-apoptin protein into cells achieved in vitro and in vivo by coupling this effector protein (henceforth referred to as the “cargo”) to a protein transduction domain. The first description of the capability of certain proteins to cross cell membranes was given independently by Green and Loewenstein (1988) and Frankel and Pabo (1988), for the HIV TAT protein. Henceforth it was shown that synthetic peptides containing the amino acids 48 to 60 derived from the HIV TAT protein could transduce into cells (Vives et al., 1997). This ability can be conferred in trans by chemically cross-linking the TAT-derived protein transduction domain to the cargo protein, enabling proteins as large as 120 kDa to be delivered intra-cellularly, in vitro as well as in vivo (Fawell et al., 1994). Other transduction domains have been described in the Antennapedia protein from Drosophila melanogaster (Derossi et al., 1998) and several synthetic peptides (Lindgren et al., 2000). The HIV TAT-mediated process does not depend on endocytosis and is not mediated through a cellular receptor. This explains the remarkable universality that is seen; the proteins can be transduced into all cells tested thus far (Schwarze and Dowdy, 2000). An efficient method based on the HIV TAT peptide has been developed to make recombinant proteins that can be transduced both in vitro and in vivo.

Significantly, when administered in vivo, all tissues, including the brain, can be targeted with a recombinant protein (Schwarze, 1999) using this system. Another class of delivery method is based on the hydrophobic core region of signal peptides (Hawiger, 1999). The cellular import of fusions or conjugations between these transduction domains and a cargo are concentration and temperature sensitive. It is, however, cell type independent, and a whole range of cells have been shown to be susceptible to internalization of cargo mediated by this class of transduction domains.

In general, for the transduction protein technology, the drawback is that it has not yet been possible to target a specific (tumor) cell compartment with this system. The lack of tumor specific targeting has thus far prevented the development of an efficient anti-tumor therapy using protein transduction.

In a preferred embodiment, the present invention describes a method that circumvents this drawback. Transduced cells, which take up TK-apoptin-derived protein, only undergo cell death when they are of a transformed or malignant nature and stay alive when they are normal. This means that the use of TK-apoptin as part of a transduction-capable proteinaceous substance will make it a potential anti-tumor agent.

The invention describes another way to introduce (recombinant) TK-apoptin fusion protein into cells, which is accomplished by fusion of the protein with a ligand. Receptor mediated internalization then results in the uptake of the fusion protein. An example for such apoptin fusion proteins are based on the Epidermal Growth Factor (“EGF”). All these methods hinge on the activity of the recombinant or purified cargo-protein in the target cell.

In particular, the invention shows that TK-apoptin protein produced in various ways retains its specific tumor killing ability and thus opens the way to combine the generalized delivery of protein transduction with the specific anti-tumor activity of apoptin and with the bystander effect of HSV-TK-ganciclovir, which results in a new method by which transformed or malignant cells can be eradicated. Thus, the invention describes several examples of TK-apoptin-derived proteinaceous substances cross-linked to a transduction domain, such as TAT, that can be applied.

Furthermore, the invention describes other transduction domains as mentioned above that can be envisaged, where all share the capacity to introduce the TK-apoptin protein into tumor cells and normal cells alike. Fusion to a ligand may specifically target TK-apoptin to one cell type, but the tumor specificity of TK-apoptin will be pivotal for therapeutic applications with minimal collateral damage to normal cells. As an example, EGF-targeted TK-apoptin could be introduced into all EGF-receptor-expressing cells. TK-apoptin will, however, destroy only the tumor cells. In addition, due to the bystander effect of TK-apoptin, tumor cells that did not contain the TK-apoptin fusion protein will undergo cell death.

The invention provides the application of cell-permeable protein as a drug, being much safer in the long term than gene therapy approaches that possibly cause genetic alterations resulting in diseases such as cancer.

The invention will be explained in more detail in the following detailed description, which is not to be construed as limiting the invention.

### BRIEF DESCRIPTION OF THE SEVERAL FIGURES OF THE DRAWINGS

In the drawings, which illustrate what is currently considered to be the best mode for carrying out the invention:

FIG. 1 is a schematic representation of Apoptin-TK fusion;

FIG. 2 is an amino acid sequence (SEQ ID NO: ) of the TK-apoptin fusion protein;

FIG. 3 is a DNA sequence (SEQ ID NO: ) of the TK-apoptin fusion protein; and

FIG. 4 is a description of the cloning strategy and the primers (primer 1 (SEQ ID NO:), primer 2 (SEQ ID NO: ), primer 3 (SEQ ID NO: ), and primer 4 (SEQ ID NO:)) used to obtain the final TK-apoptin constructs.

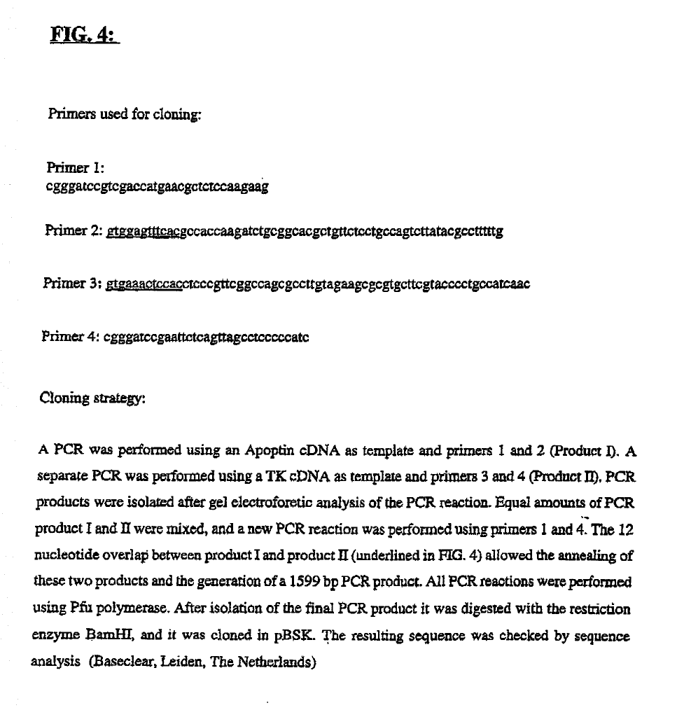

### DETAILED DESCRIPTION OF THE INVENTION

The invention includes a fusion protein comprising a polypeptide providing cytotoxicity, the polypeptide being fused to a moiety rendering the fusion protein functionally available in aberrant cells and not functionally available in non-aberrant cells. Preferably, the polypeptide provides an enzymatic activity that will convert a prodrug into a drug.

In a preferred embodiment, the polypeptide is TK and the moiety is apoptin.

The fusion protein may be conjugated to a targeting molecule for preferential delivery to cancer cells. For instance, the targeting molecule may be a liposome, folic acid, a folic acid derivative, vitamin B-12, a vitamin B-12 derivative, an antibody, an antibody fragment or a ligand for a receptor.

In a preferred method according to the invention, the method includes administering a fusion protein having a moiety rendering the fusion protein functionally available in aberrant cells and not functionally available in non-aberrant cells.

In another embodiment, the invention includes a gene delivery vehicle encoding a fusion protein comprising a moiety rendering the fusion protein functionally available in aberrant cells and not functionally available in non-aberrant cells. For instance, the gene encoding the fusion protein can encode a targeting polypeptide. In addition, the targeting polypeptide comprises a transduction domain such as TAT.

The targeting polypeptide can also comprise a member of a specific binding pair or an scFv.

The invention also includes a method for providing aberrant cells predominantly over normal cells with a desired fusion protein comprising administering a fusion protein having a moiety rendering the fusion protein functionally available in aberrant cells and not functionally available in non-aberrant cells.

Delivery vehicles can comprise adenovirus, retro, AAV, plasmovirus, nonviral polyphosphosines, lyposomes, etc.

The invention furthermore provides or describes all steps needed for the construction of a recombinant, replication-defective adenovirus expressing the TK-apoptin fusion product. High titers of recombinant-TK-apoptin adenovirus can be produced by means of adenovirus packaging cell lines, such as 293, 911 and PER.C6™. The fact that recombinant-TK-apoptin adenovirus vectors can become produced by the system described herein implies that (conditional) replicative adenovirus vector systems can become produced and used to treat cancer and autoimmune diseases.

The invention provides a system to produce and deliver intracellularly recombinant proteinaceous substances comprising TK-apoptin or functional equivalents or functional fragments thereof, or recombinant proteinaceous substances with TK-apoptin-like activity. The invention further provides for the addition of further optional modular peptides. These can include an epitope tag, allowing easy detection and immuno-precipitation without direct steric hindrance of associations of TK-apoptin with cellular proteins.

The invention provides or describes all steps needed for the production of the recombinant apoptosis-inducing agent TK-apoptin, or derivatives of TK-apoptin that have a similar tumor specificity. The recombinant protein can be produced in E. coli, insect cells by means of a baculovirus-based vector or in yeast strains (such as Pichia pastoris). The invention provides evidence that the TK-apoptin or TK-apoptin-like proteinaceous substance does not need to be folded properly in the producer cell, enabling the production of recombinant TK-apoptin or TK-apoptin-like proteinaceous substances in transgenic plant cells for mass production.

The invention proposes a modified metal affinity tag for optimal purification of the recombinant protein. By using a double His-tag next to the transduction domain, a significantly better binding to Nickel beads is achieved, resulting in the possibility to wash the recombinant TK-apoptin or TK-apoptin-like proteinaceous substances under very stringent conditions, which results in optimal purification of TK-apoptin or TK-apoptin-like proteinaceous substances.

The invention describes the use of a transduction domain fused to recombinant TK-apoptin protein. This domain allows the recombinant protein to pass through the cellular membrane. This domain can consist of a transduction domain derived from HIV TAT, or of any other known transduction domain.

The invention also provides a therapy for cancer, autoimmune diseases or related diseases, which is based on TK-apoptin or apoptin-like proteinaceous substances or virus vector systems containing the gene expressing TK-apoptin or TK-apoptin-like proteins.

The invention also provides a method to remove aberrant cells in their first stages of transformation and pre-malignant lesions, especially tumors resistant to chemotherapy.

The invention is further explained by the use of the following illustrative examples.

### EXAMPLES

Construction of the TK-Apoptin Fusion Cassette

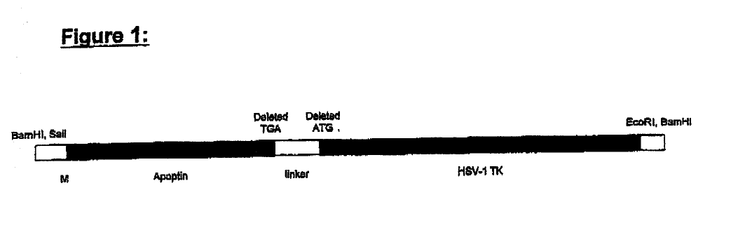

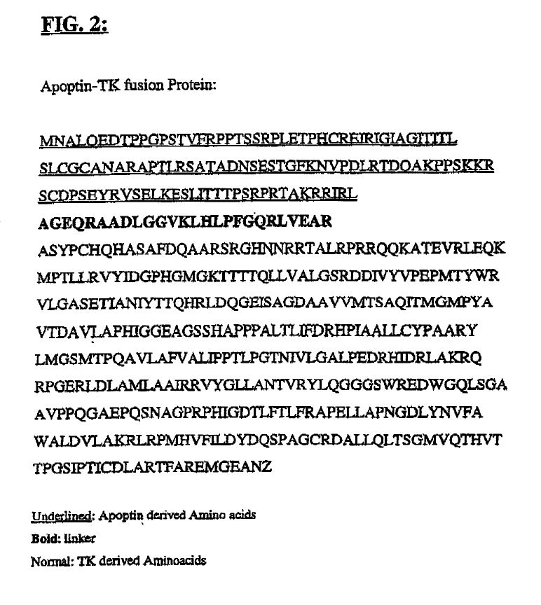

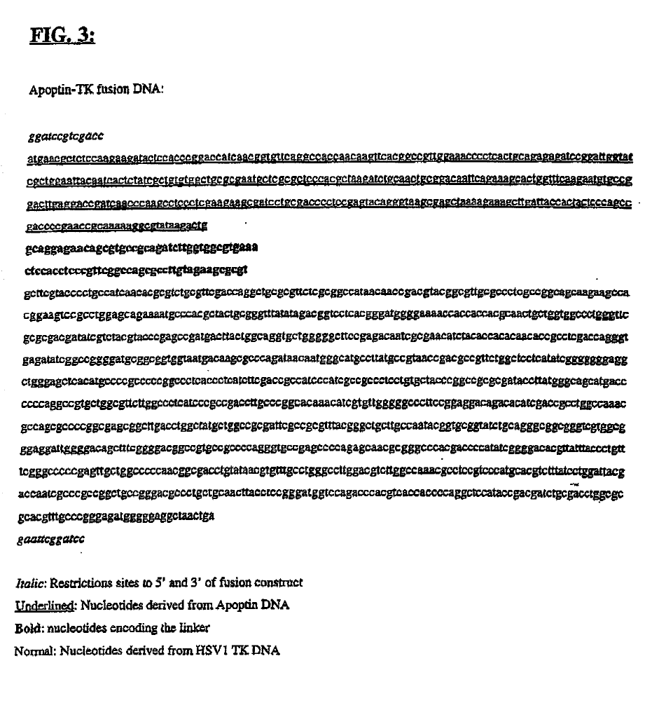

A fusion protein was constructed so that the apoptin protein was fused through a flexible linker to the N-terminus of the Thymidine Kinase protein. FIG. 1 shows a schematic representation of this construct. FIGS. 2 and 3 show the amino acid sequence (SEQ ID NO: ) and the DNA sequence (SEQ ID NO: ) of the TK-apoptin fusion product, respectively.

This construct has several features besides the presence of apoptin and HSV TK encoding regions. Upstream of the initiator ATG of the fusion protein, a BamHI restriction site was constructed to enable cloning into various expression vectors.

To expedite the cloning of this construct in a protein transduction vector, a unique SalI site was inserted that allows in-frame cloning in the pMV TAT vector, as described in the patent application filed in 2000 by Noteborn et al. (co-owned U.S. Provisional Patent Application Serial No. 60/236,117, now U.S. patent application Ser. No. 09/949,780), which is entitled “A delivery method for the tumor specific apoptosis inducing activity of apoptin”. Such a cloning would result in the addition of a transduction domain N-terminally of apoptin, and the subsequent possibility of production and purification of recombinant apoptin-TK fusion protein.

To minimize the risk of steric interference of apoptin with the TK protein, an oligonucleotide (SEQ ID NO: ) coding for a 26 amino-acid linker (SEQ ID NO: ) was inserted between the apoptin encoding ORF and the TK encoding ORF. This linker was based on a paper in which it was shown that a GFP-TK fusion with this linker is functional (Paquin et al., 2001). This linker will also reduce the possibility of negative interference of the TK protein with apoptin function. The sequence of the linker is given in FIG. 2 (SEQ ID NO:).

The start methionine of TK was replaced through the conservative substitution of leucine to minimize the risk of internal initiation at the fusion mRNA and, thus, production of TK without the apoptin moiety. To ensure the production of a full-length TK-apoptin fusion protein, the stop codon of apoptin was deleted.

Directly after the stop codon of the TK encoding ORF, an EcoRI and a BamHI restriction site were inserted to allow cloning in an expression vector or in pMV TAT.

Construction of the Transfer Vector AdApt/TK-Apoptin

The AdApt® adenoviral vector contains the cytomegalovirus (CMV) promoter, which has also been optimally adapted to the cell line PER.C6™. To examine whether it is possible to produce TK-apoptin by means of an adenovirus vector, we constructed AdApt-apoptin. To that end, the above described TK-apoptin cassette was cloned into the BamHI site of the 6.1-kb transfer vector AdApt, which was obtained from Crucell Holland, by, Leiden, NL. By sequence analysis and restriction-enzyme digestions, the correct orientation of the TK-apoptin gene under the regulation of the CMV was determined. This transfer vector has been named pAdApt/TK-apoptin. As a negative control, adenovirus transfer vector the plasmids were selected, which contain the TK-apoptin gene in the wrong orientation opposite to the CMV promoter and is named AdApt/TKAAS.

Construction and Characterization of the Adenovirus Vector AdApt/TK-Apoptin

Next, recombinant adenovirus vectors expressing the TK-apoptin gene under the regulation of the CMV promoter were generated. In addition, control adenovirus harboring the TK-apoptin gene cassette in the opposite orientation relative to the CMV promoter was made. To that end, PER.C6™ cells (Crucell Holland, by, Leiden, NL) were co-transfected with the adenovirus vector plasmid pAd5AlfII-ITR (E1−, E3+) and with the transfer plasmids pAdApt/TK-apoptin or pAdApt/TKAAS. After the observation of cytopathogenic effects of the transfected PER.C6, the medium containing the recombinant adenovirus vectors was harvested and plaque-purified (Noteborn and Pietersen, 1998).

The various plaque-purified recombinant adenovirus batches AdApt/TK-apoptin encoding the TK-apoptin and control vector AdApt/TKAAS were examined by PCR-analysis for the presence of the TK-apoptin gene in the “correct” versus “wrong” orientation, respectively (Pietersen et al., 1999). All analyzed (at least 10 in total for each vector type) recombinant adenovirus batches contained the expected TK-apoptin gene.

Replication-competent adenovirus (“RCA”) analysis by means of PCR (Pietersen et al., 1999) revealed that in all analyzed batches no RCA was generated.

Finally, the production of TK-apoptin protein by AdApt-infected human HepG2 cells was examined by means of indirect immunofluorescence using the monoclonal antibody 111.3 (Noteborn and Pietersen, 1998). The cells were almost all shown to produce TK-apoptin protein and became apoptotic very soon after infection. This was analyzed by DAPI staining as well as by means of a TUNEL assay (Pietersen, 1999). This finding is indicative of the fact that the produced TK-apoptin is completely active as an apoptotic inducer. As expected, all cells infected with AdApt/TKAAS did not stain for the monoclonal antibody and did not become apoptotic. In addition, by means of an immunoprecipitation assay and Western blot analysis using the antibody 111.3, it was shown that the expected TK-apoptin fusion product of approximately 50 kDa was detectable in the case of HepG2 cells infected with AdApt/TK-apoptin and not visible in HepG2 infected with AdApt/TKAAS or mock infected.

In conclusion, the production of TK-apoptin by means of the above described recombinant adenovirus vector system indicates that TK-apoptin can be produced in any adenoviral vector without limiting the adenovirus production.

In vitro Treatment of Human Tumor Cells with AdApt/TK-Apoptin and Ganciclovir

Next, we examined whether infection of human tumor cells with AdApt/TKapoptin and treatment with ganciclovir will result in massive cell death. To that end, human hepatoma HepG2, osteosarcoma U2OS cells, SCC-15 cells derived from squamous cell carcinoma and cells from the spontaneously transformed keratinocyte cell line HaCAT (Notebom and Pietersen, 1998) were used.

First, the various tumor cell lines were transduced with AdLacZ to determine the infective dose per cell line for obtaining 10, 30, or 50% transducibility. Subsequently, the various cell lines were infected with AdApt/TK-apoptin or AdApt/TKAAS. As positive controls, the tumor cells were infected with AdApt-apoptin (Noteborn and Pietersen, 2000), and as negative controls, cells were also infected with an adenovirus vector expressing TK (van der Eb et al., 1998) or mock infected. One day after infection, half of the cell cultures were treated with ganciclovir (5 μg per ml; refreshed after every other day).

Six days after infection, the tumor cell cultures were stained with Giemsa, as described by Notebom and Pietersen (1999). All of the tumor cell cultures that were mock infected or infected with AdTKAAS were rather viable, as well as the tumor cell cultures infected with AdTK expressing HSV thymidine kinase. At most, a slight affect on the cell viability of ganciclovir treatment was seen for the AdTKAAS- or AdTK-infected cells. The cell cultures that were for 50% transduced/infected with AdApt-apoptin or AdApt/TK-apoptin clearly showed a killing effect, which was not seen for cell cultures transduced for 50% with AdTK. These results show that apoptin (as expected) and TK-apoptin both harbor a cytotoxic effect in the absence of ganciclovir.

Upon ganciclovir treatment, the amount of cells of the four analyzed tumor cell cultures transduced for 10% with AdTK or AdTK-apoptin was strongly reduced. These data indicate that both TK and TK-apoptin have a cytotoxic effect, which is mainly mediated by a bystander effect. The HepG2 cells showed the best bystander effect upon TK-apoptin treatment. At a transduction rate of 10%, almost all HepG2 cells were dead.

Treatment of Normal Rat Hepatocytes

The most important drawback of the adenovirus-TK/ganciclovir system is the dramatic affect on the viability of normal hepatocytes as reported, for example, by van der Eb et al., 1998. Therefore, we tested the effect of AdApt/TK-apoptin in comparison with AdTK on rat hepatocytes. To that end, cultures with freshly isolated rat hepatocytes were infected with AdTk or AdApt/TK-apoptin and treated with ganciclovir. By means of indirect immunofluorescence assays, which were carried out in parallel, it was determined that about 5-10% of the normal rat liver cells were producing TK or TK-apoptin, respectively. As a negative control, the rat hepatocytes were mock infected.

One day after infection, ganciclovir was added as described above for the human tumor cell lines. Four days after infection, the cells were stained with Giemsa. The amount of hepatocytes infected with AdTK or AdApt/TK-apoptin was similar to those that were mock infected. The hepatocyte cultures that were infected with AdTK and treated with ganciclovir, however, were strongly reduced in the amount of hepatocytes. In contrast, hepatocytes infected with AdApt/TK-apoptin and treated with ganciclovir only showed a slight reduction of the amount of hepatocytes.

These data clearly show that TK-apoptin can induce cell death in tumor cells, which contain TK-apoptin product and also in tumor cells that were not transduced, by means of a bystander effect. More importantly, normal (rat) hepatocytes, which are killed by AdTK/ganciclovir treatment, are viable upon AdApt/TK-apoptin and ganciclovir treatment. The apoptin tumor-specific apoptosis characteristics seem to hold also in the setting of TK-apoptin fusion protein delivered by an adenoviral vector.

Most likely, apoptin harbors an activity that can sequester the TK-apoptin fusion products in cytoplasmic structures of normal human cells, which also has been described for MBP-apoptin fusion product in co-owned U.S. Provisional Patent Application Serial No. 60/236,117, now U.S. patent application Ser. No. 09/949,780, by Noteborn et al. entitled “A delivery method for the tumor-specific apoptosis inducing activity of apoptin”.

Treatment of Human RA-Derived Fibroblast-Like Synoviocytes and Normal Fibroblast Synoviocytes with AdApt/TK-Apoptin

Next, we tested the activity of AdApt/TK-apoptin in RA-derived fibroblast synoviocytes versus ones derived from healthy persons (material was obtained from the Rheumatology Department, Leiden University Medical Center, Leiden, NL). As a positive control, cells were infected with AdApt-apoptin or AdTk, and as a negative control, cells were mock infected. In parallel immunofluorescence analysis showed that about 5-10% of the cells were infected. Harvesting of the cells and Giemsa staining was carried out as described above.

Upon ganciclovir treatment, both normal as well as RA-derived cells treated with AdTK underwent significant cell death, which was the case only for RA-derived cells treated with AdApt/TK-apoptin and not for the cells derived from healthy persons. All the other cell cultures did not reveal a visible reduction of cell amount.

These data imply that TK-apoptin also has a specific cell-killing activity in RA-related cells, which is at least dramatically reduced in normal cells derived from healthy individuals.

Production of Recombinant TK-Apoptin Protein

The production and purification of recombinant MBP-TK-apoptin protein has been carried out in an E. coli system as has been described for MBP-apoptin by Noteborn et al. in co-owned U.S. Provisional Patent Application Serial No. 60/236,117, now U.S. patent application Ser. No. 09/949,780.

Tumor-Specific Induction of Apoptosis

To assay the ability of MBP-TK-apoptin protein to specifically induce apoptosis in tumor cells, a microinjection system was set up. Human osteosarcoma Saos-2 cells and normal human VH10 fibroblasts were cultured on glass cover slips. The cells were microinjected in the cytoplasm with MBP-TK-apoptin protein or as a positive control MBPapoptin or MBP using an Eppendorf microinjector with an injection pressure of 0.5 psi. The cells were co-injected with dextran-rhodamine to be able to later identify the microinjected cells.

The cells were fixed and analyzed by using the methods described by Noteborn et aL in co-owned U.S. Provisional Patent Application Serial No. 60/236,117, now U.S. patent application Ser. No. 09/949,780.

The results show that both MBP-apoptin and MBP-TK-apoptin were able to induce rapid apoptosis in human tumor cells (within 3-6 hours) but did not induce apoptosis in normal human cells. The MBP control protein did not induce apoptosis in any of the cell preparations tested under these conditions. Remarkably, the apoptin fusion proteins were present in the nucleus of tumor cells but sequestered in cytoplasmic structures within the VH10 cells.

The following observations may help to further explain the tumor-specific activity of MBP-apoptin and MBP-TK-apoptin. Twenty-four hours after microinjection, almost all tumor cells (apoptotic at this time point) contained MBP-apoptin or MBP-TK-apoptin. The tumor cells (viable) were positive for MBP upon microinjection of MBP protein. The VH10 cells microinjected with MBP-apoptin or MBP-TK-apoptin, however, were negative when stained for MBP or apoptin but still contained dextran-rhodamine. In contrast, VH10 cells microinjected with MBP protein still contained MBP and the MBP-signal was not reduced in comparison with MBP-microinjected cells that were harvested after 1 or 3 hours.

These data show that the recombinant proteinaceous substance TK-apoptin can induce apoptosis in tumor cells and not in normal non-transformed cells. This implies that TK does not interfere with the tumor-specific induction of apoptosis by apoptin and is inactivated by proteolytic degradation or shielding.

To explore this issue further, we decided to use a biochemical approach. We had already noticed that transfected apoptin could be readily immunoprecipitated with stringent RIPA buffer, but not at all in mild buffer (Noteborn and Rohn, unpublished observations), suggesting that normal-specific epitope shielding was the more likely possibility. With this in mind, we decided to evaluate epitope shielding by immunoprecipitation compared with the amount of protein actually present in the lysate without immunoprecipitation.

First, we seeded VH10 cells and Saos-2 cells and the next day microinjected MBP-apoptin protein or MBP-TK-apoptin protein into the cytoplasm of 500 cells per dish. At 1, 6 and 24 hours following microinjection, cell lysates were made with mild lysis buffer. The supernatant of each clarified lysate was split into equal aliquots, resuspended in sample buffer, resolved by SDS-PAGE, and subjected to Western blot analysis. The recombinant MBP-apoptin and MBP-TK-apoptin in one aliquot was detected by antibodies against the C-terminal region of apoptin (VP3-C) and in another by antibodies directed against MBP. Non-injected cell lysate was analyzed in parallel for negative control.

MBP-apoptin or MBP-TK-apoptin could be readily immunoprecipitated from injected tumor cells at both time points, as assessed by VP3-C staining on the Western blot. However, in normal cells MBP-TK-apoptin or MBP-apoptin was not pulled down efficiently, even at one hour after microinjection, and after 6 hours, it could not be immunoprecipitated at all. This phenomenon was also seen when the Western blot was reprobed with an antibody against MBP, although the shielding was not as marked here.

This result suggests that the entire molecule might not be readily available under native conditions, perhaps due to its aggregation capability.

Next, VH10 cells were microinjected with MBP-apoptin protein or MBP-TKapoptin protein (of each 500 cells per dish), and lysed at 3 h (early time) and 24 h (late time) after microinjection with mild lysis buffer versus the strongly denaturing RIPA lysis buffer. Each lysate supernatant of identical cells was equally divided into two aliquots. One aliquot was immunoprecipitated with VP3C antibodies. Another aliquot was directly added into sample buffer for Western blotting without immunoprecipitation. Meanwhile, non-injected cell lysates were made with both lysis buffers as controls. All samples were separated on parallel 12.5% SDS-PAGE and electroblotted. Blots were incubated with the antibody MBP-probe and probed with ProtA-HRP for immunoprecipitated samples and GAR-HRP for non-immunoprecipitated samples. Without immunoprecipitation, MBP-apoptin or MBP-TK-apoptin from the lysate made with either mild lysis buffer or RIPA lysis buffer was equally detected by antibodies directed against MBP at both early (3 h) and late (24 h) time points. However, the apoptin-specific antibody did not strongly immunoprecipitate MBP-apoptin or MBP-TK-apoptin from the lysate made with mild lysis buffer, allowing only very weak detection of MBP-apoptin or MBP-TK-apoptin at the early time point (3 h) and a complete failure to detect at the late time point (24 h). In contrast, VP3C antibodies efficiently immunoprecipitated MBP-apoptin or MBP-TK-apoptin from the lysate made with RIPA lysis buffer at both early (3 h) and late (24 h) time points.

These results strongly suggest that the negative detection of MBP-apoptin in normal cells is due to the epitope shielding on the apoptin moiety of the protein, a shielding which grows more intense with time post-injection.

To determine the long-term fate of MBP-apoptin and MBP-TK-apoptin in normal human cells, we again microinjected VH10 cells with MBP-apoptin protein, MBP-TK-apoptin protein, MBP protein or Rho-Dex alone, as described above. Cells were lysed in the stringent RIPA-buffer at 1, 2, 3, 4 and 5 days after microinjection and analyzed by Western blotting using antibodies directed against apoptin or MBP, as mentioned above. Both MBP and Rho-Dex staining was still prevalent after 5 days, whereas MBP-apoptin or MBP-TK-apoptin disappeared completely after 2 days.

These data clearly show that MBP-TK-apoptin and MBP-apoptin, due to normal-specific features, become degraded.

The fact that apoptin enables degradation of the fusion protein of which it is part, is further demonstrated by the following microinjection and fluorescence-microscope experiments. MBP-apoptin was labeled with fluorescein (kind gift of Rutger Leliveld, University of Leiden, Leiden, The Netherlands). Microinjection of Saos-2 cells with the fluorescein-labeled MBP-apoptin protein resulted in induction of apoptosis in a similar rate as was described for MBP-apoptin that was not labeled with fluorescein. Furthermore, fluorescein-labeled apoptin was detectable in these tumor cells for at least 2 days after microinjection.

Microinjection of fluorescein-labeled MBP-apoptin in VH10 cells resulted in the sequestering of labeled MBP-apoptin in the same cytoplasmic structures as observed for MBP-apoptin. After 24 hours, fluorescence microscope analysis showed that almost all microinjected VH10 cells did not contain detectable amounts of fluorescein-labeled MBP-apoptin.

These data clearly show that in normal cells, apoptin (fusion protein) becomes sequestered in cytoplasmic structures and subsequently becomes degraded and, thus, functionally inactivated. These data are also confirmed by observations in our laboratory that the level of apoptin protein in normal splenocytes derived from transgenic-apoptin mice increases dramatically upon addition of proteasomal inhibitors (Pietersen and Noteborn, personal communication).

MBP-Apoptin and MBP-TK-Apoptin do not Induce Apoptosis in Other Human Primary Cells

Because certain human primary cell types are known to be more sensitive than fibroblasts to the effects of standard tumor therapy, such as chemo or radiation regimens, we decided to exploit the utility of the microinjection technique to circumvent problems of poor transfection efficiency and determine whether apoptin was toxic in these more unusual cells. To this end, we microinjected human primary hepatocytes and human primary mesenchymal stem cells with MBP-apoptin protein, MBP-TK-apoptin protein and MBP protein. As a positive control to confirm that the cells were competent to undergo apoptosis, we also microinjected a plasmid encoding the death-effector FADD (Chinnaiyan and Dixit, 1996) into the nucleus of these cells at one time point.

Both cell types, although exquisitely sensitive to FADD-induced apoptosis, remained resistant to the effects of MBP-apoptin and MBP-TK-apoptin. Instead, MBP-apoptin and MBP-TK-apoptin remained harmlessly in the cytoplasm and formed cytoplasmic aggregation structures, as observed above for VH10 cells. Twenty-four hours after microinjection with MBP-apoptin or MBP-TK-apoptin, almost all human hepatocytes and mesenchymal stem cells were negative when stained for MBP or apoptin but still contained dextran-rhodamine, as has also been seen for VH10 cells. In contrast, human hepatocytes and mesenchymal stem cells microinjected with MBP protein still contained clearly detectable amounts of MBP.

Next, we wanted to evaluate one of the most sensitive of all cell types, CD34+ bone marrow stem cells. Because these cells are grown in suspension, we first affixed them to the microinjection plate using a coating of Wheat Germ Agglutinin because this cell type is known to possess many receptors for this lectin. Cells were cultured overnight in medium tailored to retain their primitive characteristics. We were unable to microinject the limited cytoplasmatic area of this tiny cell type with the smallest commercially available needles, so we used a plasmid encoding wild-type apoptin or TK-apoptin to perform the test compared with a plasmid encoding the non-apoptotic desmin gene as a negative control and FADD as a positive control. Although FADD was highly toxic in this experiment, apoptin or TK-apoptin did not induce above-background levels of apoptosis.

Thus, gene or protein therapy with TK-apoptin is likely to have a larger therapeutic window due to low side effects in normal tissues, even especially sensitive ones.

Prevention of Tumor Cell Growth in vitro by TAT-TK-Apoptin

The production of MBP-TK-apoptin-TAT protein has been carried out in the same vectors, production systems and purification methods as has been described for MBP-apoptin(vp3)-TAT protein in co-owned U.S. Provisional Patent Application Serial No. 60/236,117, now U.S. patent application Ser. No. 09/949/780.

To examine whether MBP-TK-apoptin-TAT can specifically cause apoptosis in human tumor cells, Saos-2 and U20S (tumor) and VH10 (normal) cells were split 1:10 and cultured for 1 week in the presence of 1 μg/ml MBP-TK-apoptin-TAT protein or MBP-apoptin-TAT, or in normal medium. Half of the cultures were treated with ganciclovir, as described above, from day 2 onward. Subsequently, the cells were fixed with methanol-acetic acid and stained with Coomassie Blue. Although the cells in the control medium had all grown to confluency, the Saos-2 and U20S cells treated with MBP-TK-apoptin-TAT or MBP-apoptin-TAT had undergone significant apoptosis. The cells treated with MBP-TK-apoptin-TAT protein and ganciclovir completely disappeared from the dish. The VH10 cells treated with MBP-apoptin-TAT or MBP-TK-apoptin-TAT, in the vicinity of ganciclovir or not, had all also grown to the same density as control treated VH10 cells, showing that MBP-TK-apoptin-TAT does not affect the growth of non-transformed cells.

### REFERENCES

Aghi, M., Hochberg, and Breakefield, X. O. (2000), “Prodrug activation enzymes in cancer gene therapy”, J. Gene Medicine 2, 148-164.

Aupperle, K. R., Boyle, D. L., Hendrix, M., Seftor, E. A., Zvaifler, N. J., Barbosa, M., and Firestein (1998), “G. S. Regulation of synoviocyte proliferation, apoptosis, and invasion by the p53 tumor-suppressor gene”, American J. Pathology 152, 1091-1098.

Bellamy, C. O. C., Malcomson, R. D. G., Harrison, D. J., and Wyllie, H. (1995), “Cell death and disease: The biology and regulation of apoptosis”, Seminars in Cancer Biology 6, 3-12.

Bucala, R., Ritchlin, C., Winchester, R., Cerami, A. (1991), “Constitutive production of inflammatory and mitogenic cytokines by rheumatoid synovial fibroblasts”, J. Experimental Medicine 173, 569-574.

Chinnaiyan, A. M. and Dixit, V. (1996), “Induction of apoptosis by tumor suppressor genes and oncogenes”, Seminars in Cancer Biology 6: 17-25.

Danen-Van Oorschot, A. A. A. M., Fischer, D. F., Grimbergen, J. M., Klein, B., Zhuang, S. -M., Falkenburg, J. H. F., Backendorf, C., Quax, P. H. A., Van der Eb, J. A., and Notebom, M. H. M. (1997). Apoptin induces apoptosis in human transformed and malignant cells but not in normal cells. Proceedings National Academy Sciences, USA: 94, 5843-5847.

Danen-Van Oorschot et al. (1997a), “BAG-I inhibits p53-induced but not apoptin-induced apoptosis”, Apoptosis 2, 395-402.

Derossi, D., Chassaing, G., and Prochiantz, A. (1998), “Trojan peptides: The penetration system for intracellular delivery”, Trends in Cell Biology 8, 84-87.

Duke, R. C., Ocjius, D. M., Young, J. D-E. (1996), “Cell suicide in health and disease”, Scientific American, December 1996, 48-55.

Earnshaw, W. C. (1995), “Nuclear changes in apoptosis”, Current Opinion in Cell Biology 7, 337-343.

Fels, C., Schafer, C., Huppe, B., Bahn, H., Heidecke, V., Kramm, C. M., Lautenschlager, C., and Rainov, N. G. (2000), “Bcl-2 expression in higher-grade human glioma: a clinical and experimental study”, J. of Neurooncology 48, 207-216.

Fawell, S., Seery, J., Daikh, Y., Moore, C., Chen, L., Pepinsky, S., and Barscum, J., “Tatmediated delivery of heterologous proteins into cells”, Proc. Nat'l Acad. Sci. USA 91, 664-668.

Firestein, G. S. (1995), “Invasive fibroblast-like synoviocytes in rheumatoid arthritis”, Arthritis and Rheumatism 39, 1781-1790.

Firestein, G. S. (1995a), “Apoptosis in rheumatoid arthritis synovium”, J. Clinical Investigations 96, 1631-1638.

Firestein, G. S. (1997), “Rheumatoid arthritis: Etiology and Pathogenesis of Rheumatoid arthritis”, Textbook of Rheumatology, 5th edition, Eds. Kelley, Harris, Ruddy and Sledge, Chapter 54, pp. 851-897.

Firestein, G. S. (1998), “Novel therapeutic strategies involving animals, arthritis, and apoptosis”, Current Opinion in Rheumatology 10, 236-241.

Frankel, A. D., and Pabo, C. O. (1988), “Cellular uptake of the Tat protein from human immunodeficiency virus”, Cell 55, 1189-1193.

Gay, S. and Gay, R. E. (1989), “Cellular basis and oncogene expression of rheumatoid joint destruction”, Rheumatology International 9, 105-113.

Geiler, T., Kriegsmann, J., Keyzer, G. M., Gay, R. E., Gay, S. (1994), “A new model for rheumatoid arthritis generated by engraftment of rheumatoid synovial tissue and normal human cartilage into SCID mice”, Arthritis Rheumatology 37, 1664-1671.

Green, M., and Loewenstein, P. M. (1988), “Autonomous functional domains of chemically synthesized human immunodeficiency virus Tat trans-activator protein”, Cell 55, 11791188.

Hawiger, J. (1999), “Non-invasive intracellular delivery of functional peptides and proteins”, Current Opinion in Chemical Biology 3, 89-94.

Hockenberry, D. M. (1994), “Bcl-2 in cancer, development and apoptosis”, J. of Cell Science (Supplement) 18, 51-55.

Kerr, J. F. R., Winterford, C. M., and Harmon, B. V. (1994), “Apoptosis: Its significance in cancer and cancer therapy”, Cancer 73, 2013-2026.

Lafyatis et al. (1998), “Anchorage-independent growth of synoviocytes from arthritic and normal joints: stimulation by exogenous platelet-derived growth factor and inhibition of by transforming growth factor beta and retinoids”, J. Clinical Investigations 83, 1267-1276.

Levine, A. J. (1997), “p53, the cellular gatekeeper for growth and division”, Cell 88, 323-331.

Lindgren, M., Haelbrink, M., Prochiantz, A., and Langel, U. (2000), “Cell penetrating peptides”, Trends in Pharmacological Sciences 21, 99-103.

McDonell T. J., Meyn, R. E., Robertson, L. E. (1995), “Implications of apoptotic cell death regulation in cancer therapy”, Seminars in Cancer Biology 6, 53-60.

Noteborn, M. H. M. (1996), International Patent Publication WO 96/41191, “Apoptin induces apoptosis in human transformed and malignant cells but not in normal cells as essential characteristic for the development of an anti-tumor therapy”.

Noteborn, M. H. M., and De Boer, G. F. (1996) U.S. patent application Ser. No. 030,335.

Noteborn, M. H. M. and Pietersen, A. M. (2000), Reuma. PCT/NL00/00013.

Notebom, M. H. M., De Boer, G. F., Van Roozelaar, D., Karreman, C., Kranenburg, O., Vos, J., Jeurissen, S., Zantema, A., Hoeben, R., Koch, G., Van Ormondt, H., and Van der Eb, A. J. (1991), “Characterization of cloned chicken anemia virus DNA that contains all elements for the infectious replication cycle”, J. of Virology 65, 3131-3139.

Noteborn, M. H. M., and Pietersen, A. (1998), PCT Patent Application No. PCT/NL98/00213, “A gene delivery vehicle expressing the apoptosis-inducing proteins VP2 and/or apoptin”.

Noteborn, M. H. M., Todd, D., Verschueren, C. A. J., De Gauw, H. W. F. M., Curran, W. L., Veldkamp, S., Douglas, A. J., McNulty, M. S., Van der Eb, A. J., and Koch, G. (1994), “A single chicken anemia virus protein induces apoptosis”, J. of Virology 68, 346-351.

Noteborn, M. H. M., Verschueren, C. A. J., Koch, G., and Van der Eb, A. J. (1998), “Simultaneous expression of recombinant baculovirus-encoded chicken anemia virus (CAV) proteins VP1 and VP2 is required for formation of the CAV-specific neutralizing epitope”, J. General Virology 79, 3073-3077.

Noteborn, M. H. M., and Zhang, Y. (1998), PCT Patent Application No. PCT/NL98/00457, “Methods and means for determining the transforming capability of agents, for determining the predisposition of cells to become transformed and prophylactic treatment of cancer using apoptin-like activity”.

Noteborn et al. (1998a), “Chicken anemia virus: Induction of apoptosis by a single protein of a single-stranded DNA virus”, Seminars in Virology 8, 497-504.

Paquin, A., Jaalouk, D. E., Galipeau, J. (2001), “Retrovector encoding a green fluorescent protein-herpes simplex virus thymidine kinase fusion protein serves as a versatile suicide/reporter for cell and gene therapy applications”, Human Gene Therapy 12,13-23.

Paulovich, A. G., Toczyski, D., Hartwell, H. (1997), “When checkpoints fail”, Cell 88, 315-321.

Pietersen, A. M., Van der Eb, M. M., Rademaker, H. J., Van den Wollenberg, D. J. M., Rabelink, M. J. W. E., Kuppen, P. J. K., Van Dierendonck, J. H., Van Ormondt, H., Masman, D., Van de Velde, C. J. H., van der Eb, Hoeben, R. C., and Notebom, M. H. M. (1999), “Specific tumor-cell killing with adenovirus vectors containing the apoptin gene”, Gene Therapy 6, 882-892.

Remmers, E. F., Lafyatis, R., Kumkumian, G. K., Case, J. P., Roberts, A. B., Spom, M. B., and Wilder, R. L. (1990), “Cytokines and growth regulation of synoviocytes from patients with rheumatoid arthritis and rats with streptococcal cell wall arthritis”, Growth Factors 2, 179-188.

Sachs, L. and Lotem, J. (1993), “Control of programmed cell death in normal and leukemia cells: New implications for therapy”, Blood 82, 15-21.

Steller, H. (1995), “Mechanisms and genes of cellular suicide”, Science 267, 1445-1449.

Schwarze, S. R., Ho, A., Vocero-Akbani, A., and Dowdy, S. (2000), “In vivo protein transduction: delivery of a biologically active protein into the mouse”, Science 285, 1569-1572.

Teodoro, J. G. and Branton, P. E. (1997), “Regulation of apoptosis by viral gene products”, J. of Virology 71, 1739-1746.

van der Eb et al. (1998), “Severe hepatic dysfunction after adenovirus-mediated transfer of the Herpes Simplex thymidine-knase gene and ganciclovir administration”, Gene Therapy 5, 451-458.

Vives, E., Brodin, P., and Lebbleu, B. (1997), “A truncated HIV-1 Tat protein basic domain rapidly translocates through the plasma membrane and accumulates in the cell nucleus”, J. of Biological Chemistry 272, 16010-16017.

White, E. (1996), “Life, death, and the pursuit of apoptosis”, Genes and Development 10, 1-15.

Wyllie, A. H. (1995), “The genetic regulation of apoptosis”, Current Opinion in Genetics and Development 5, 97-104.

Wyllie, A.H., Kerr, J.F.R., Currie, A.R. (1980), “Cell death: The significance of apoptosis”, International Review of Cytology 68, 251-306.

Zhuang, S. -M., Landegent, J. E., Verschueren, C. A. J., Falkenburg, J. H. F., Van Ormondt, H., Van der Eb, A. J., Notebom, M. H. M. (1995), “Apoptin, a protein encoded by chicken anemia virus, induces cell death in various human hematologic malignant cells in vitro”, Leukemia 9 S1, 118-120.

Zhuang, S. -M., Shvarts, A., Van Ormondt, H., Jochemsen, A. -G., Van der Eb, A. J., Noteborn, M. H. M. (1995), “Apoptin, a protein derived from chicken anemia virus, induces a p53-independent apoptosis in human osteosarcoma cells”, Cancer Research 55, 486-489.

What is claimed is:

1. A fusion protein comprising a polypeptide providing cytotoxicity, said polypeptide being fused to a moiety rendering the fusion protein functionally available in aberrant cells and not functionally available in non-aberrant cells.

2. The fusion protein of claim 1, wherein the polypeptide provides enzymatic activity that converts a prodrug into a drug.

3. The fusion protein of claim 1, wherein said polypeptide is TK.

4. The fusion protein of claim 1, wherein said moiety is apoptin.

5. The fusion protein of claim 1, wherein said fusion protein is conjugated to a targeting molecule.

6. The fusion protein of claim 5, wherein the targeting molecule is selected from the group consisting of liposomes, folic acid and folic acid derivatives, vitamin B-12 and vitamin B-12 derivatives, an antibody or antibody fragment, and a ligand for a receptor.

7. A method of inducing cell death in aberrant cells, said method comprising:
administering a fusion protein having a moiety rendering the fusion protein functionally available in aberrant cells and not functionally available in non-aberrant cells.

8. A gene delivery vehicle encoding a fusion protein comprising a moiety rendering the fusion protein functionally available in aberrant cells and not functionally available in non-aberrant cells.

9. The gene delivery vehicle of claim 8, wherein the gene encoding the fusion protein encodes a targeting polypeptide.

10. The gene delivery vehicle of claim 9, wherein the targeting polypeptide comprises a transduction domain.

11. The gene delivery vehicle of claim 10, wherein said transduction domain comprises TAT.

12. The gene delivery vehicle of claim 9, wherein the targeting polypeptide comprises a member of a specific binding pair.

13. The gene delivery vehicle of claim 9, wherein the targeting polypeptide comprises an scFv.

14. The gene delivery vehicle of claim 8, wherein the gene encoding the fusion protein encodes two proteins.

15. A method for providing aberrant cells predominantly over normal cells with a desired fusion protein comprising administering a fusion protein having a moiety rendering the fusion protein functionally available in aberrant cells and not functionally available in nonaberrant cells.
